(19) United States
(12) Patent Application Publication
(10) Pub. No.: US 2014/0276189 A1
(43) Pub. Date: Sep. 18, 2014

(54) STATE DETERMINATION DEVICE, STATE DETERMINATION METHOD, AND RECORDING MEDIUM STORING STATE DETERMINATION PROGRAM

(71) Applicant: FUJITSU LIMITED, Kawasaki-shi (JP)

(72) Inventor: KOUICHIROU KASAMA, Kawasaki (JP)

(73) Assignee: FUJITSU LIMITED, Kawasaki-shi (JP)

(21) Appl. No.: 14/180,546

(22) Filed: Feb. 14, 2014

(30) Foreign Application Priority Data
Mar. 18, 2013 (JP) 2013-055114

(51) Int. Cl. A61B 5/22; A61B 5/00; A61B 5/11; A61B 5/0492; A61B 5/04

(52) U.S. Cl. CPC A61B 5/222; A61B 5/0492; A61B 5/04012; A61B 5/11; A61B 5/742; A61B 5/7475; A61B 5/0004
USPC 600/546

(57) ABSTRACT

A state determination device includes a myopotential measurement unit configured to measure a myopotential of a surface of the body, a myopotential integrated value calculation unit configured to calculate a myopotential integrated value by integrating, with time, the myopotential measured by the myopotential measurement unit for a measurement period at predetermined measurement intervals, and a fatigue determination unit configured to determine presence/absence of fatigue of the body based on a size of the myopotential integrated value calculated by the myopotential integrated value calculation unit, and a change amount of the myopotential integrated value.

### CROSS-REFERENCE TO RELATED APPLICATION

This application is based upon and claims the benefit of priority of the prior Japanese Patent Application No. 2013-055114, filed on Mar. 18, 2013, the entire contents of which are incorporated herein by reference.

### FIELD

The embodiments discussed herein are related to a state determination device, a state determination method, and a state determination program, which determine a state of the body.

### BACKGROUND

Methods for determining presence/absence of fatigue of the body or an aerobic exercise are conventionally known. For example, fatigue or an aerobic exercise can be determined by analyzing components of blood. In this case, however, it is needed to draw blood from the body and analyze the blood by a dedicated research institute. This analysis sometimes needs several days, posing problems such that a physical and financial burden is imposed to determine fatigue or an aerobic exercise, and it is difficult to measure fatigue or an aerobic exercise in real time.

Related to the above described techniques, a method for obtaining an anaerobic threshold of a user by measuring heartbeats of the user when he or she does a physical exercise while changing an exercise load gradually or in stages, and by analyzing results of the measurement with a specified method is known.

Additionally, a muscle fatigue evaluation device for calculating an integrated value by processing, as absolute values, a time series signal having a gradient of a power value obtained by performing a specified operation for signal data obtained from a myopotential detection sensor, and a time series signal having a gradient of the largest Lyapunov exponent, and for using the integrated value as the degree of fatigue (the progress of fatigue) is known.

Furthermore, an exercise machine for estimating an anaerobic threshold based on an electro-cardiographic signal in response to a load change of a load device, and for controlling a load based on the estimated value is known.

### DOCUMENTS OF PRIOR ART

### Patent Documents

Patent Document 1: Japanese Laid-open Patent Publication No. 2007-075172

Patent Document 2: Japanese Laid-open Patent Publication No. 2007-209453

Patent Document 3: National Publication of International Patent Application No. WO/1999/043392

### SUMMARY

According to an aspect of the embodiments, a state determination device includes the following components.

A myopotential measurement unit measures a myopotential of a surface of the body.

A myopotential integrated value calculation unit calculates a myopotential integrated value by integrating, with time, the myopotential measured by the myopotential measurement unit for a measurement period at predetermined measurement intervals.

A fatigue determination unit determines presence/absence of fatigue of the body based on a size of the myopotential integrated value calculated by the myopotential integrated value calculation unit, and a change amount of the myopotential integrated value.

The object and advantages of the invention will be realized and attained by means of the elements and combinations particularly pointed out in the claims.

It is to be understood that both the foregoing general description and the following detailed description are exemplary and explanatory and are not restrictive of the invention.

### BRIEF DESCRIPTION OF DRAWINGS

FIG. 1 is an explanatory diagram of a state determination device according to an embodiment;

FIG. 2 illustrates a configuration example of a portable terminal device for implementing the state determination device, according to another embodiment;

FIG. 3 is an external view of the portable terminal device;

FIG. 4 is a flowchart illustrating a state determination process executed by the portable terminal device according to the embodiment;

FIG. 5 is a flowchart illustrating the state determination process executed by the portable terminal device according to the embodiment;

FIG. 6 illustrates time characteristics of a myopotential integrated value at the time of an aerobic exercise;

FIG. 7 illustrates a relationship between an acceleration and an exercise intensity;

FIG. 8 illustrates a display example of process results in steps S410 to S413;

FIG. 9 illustrates a display example of process results in steps S414 to S417;

FIG. 10 illustrates another configuration example of a myopotential sensor;

FIG. 11 illustrates a further configuration example of the myopotential sensor; and

FIG. 12 illustrates a configuration of an information processing device.

### DESCRIPTION OF EMBODIMENTS

Embodiments are described in detail below with reference to FIGS. 1 to 11. The following embodiments are merely examples, and are not intended to preclude various modifications and technical applications, which are not explicitly described below. Namely, the embodiments may be variously modified and implemented, for example, by combining techniques recited as embodiments within a scope that does not depart from the gist of the embodiments. Moreover, process procedures represented by the flowcharts in FIGS. 4 and 5 are not intended to limit the order of processes. Accordingly, the order of processes may be naturally changed if possible.

### Embodiment

FIG. 1 is an explanatory diagram of a state determination device 100 according to an embodiment.

The state determination device 100 includes a myopotential measurement unit 110, a myopotential integrated value calculation unit 120, and a fatigue determination unit 130. Note that the myopotential integrated value calculation unit 120 and the fatigue determination unit 130 may be implemented by causing a CPU, which is included in the state determination device 100 and not illustrated, to execute a specified program.

The myopotential measurement unit 110 measures a myopotential of a surface of the body 150. As the myopotential measurement unit 110, a myopotential sensor or the like is available.

The myopotential integrated value calculation unit 120 calculates a myopotential integrated value by integrating, with time, the myopotential measured by the myopotential measurement unit 110 for a measurement period at predetermined measurement intervals.

The fatigue determination unit 130 determines presence/absence of fatigue of the body 150 based on a size of the myopotential integrated value calculated by the myopotential integrated value calculation unit 120 and a change amount of the myopotential integrated value. For example, the fatigue determination unit 130 calculates the change amount of the myopotential integrated value calculated by the myopotential integrated value calculation unit 120 at this time from the myopotential integrated value calculated by the myopotential integrated value calculation unit 120 at preceding time. Then, the fatigue determination unit 130 can determine that the body 150 is fatigued if the myopotential integrated value is equal to or smaller than a threshold value X and the calculated change amount of the myopotential integrated value is equal to or smaller than a threshold value Y. This adopts the phenomenon that myopotential actions are reduced when the body 150 is getting fatigued, leading to a decrease in the value of a myopotential. Note that a predetermined positive real value is available as the threshold value X. Moreover, a predetermined real value is available as the threshold value Y.

As described above, the state determination device 100 determines the presence/absence of the fatigue of the body 150 based on the size of the myopotential integrated value calculated by integrating, with time, the myopotential measured from the body 150 for a measurement period, and the change amount of the myopotential integrated value. Accordingly, with the state determination device 100, a state of the body can be easily determined without performing operations such as analyzing components of blood after drawing the blood from the body 150.

### Another Embodiment

FIG. 2 illustrates a configuration example of a portable terminal device 200 that implements the state determination device, according to another embodiment.

The portable terminal device 200 includes a CPU (Central Processing Unit) 201, a myopotential sensor 202, an acceleration sensor 203, a subprocessor 204, a memory 205, a flash memory 206, an LCD (Liquid Crystal Display) 207, and a touch panel 208.

The CPU 201 is an arithmetic processing unit for executing a program for implementing a state determination process according to this embodiment in addition to execution of peripheral devices and various types of software. For example, the CPU 201 executes the state determination process illustrated in FIGS. 4 and 5 according to a program expanded in the memory 205.

The CPU 201 calculates a myopotential integrated value by integrating, with time, a myopotential measured by the myopotential sensor 202 per unit time, for one minute in this embodiment. Then, the CPU 201 determines whether or not the body of a user of the portable terminal device 200 is doing an aerobic exercise based on the myopotential integrated value per unit time and a change of the value.

This embodiment can adopt the phenomenon that a myopotential increases with time if an aerobic exercise is continued with a certain exercise intensity. For instance, in the example of FIG. 6, a myopotential integrated value is constant when an exercise is done with an exercise intensity of 1.5 [Mets] or 2.0 [Mets]. In contrast, when an exercise is done with an exercise intensity of 2.5 [Mets] or higher, the myopotential integrated value increases. As described above, by detecting an increase in the myopotential integrated value when an exercise is done with a certain exercise intensity, the CPU 201 can determine that the body of a user of the portable terminal device 200 is doing an aerobic exercise. In this embodiment, the CPU 201 determines that the body of the user of the portable terminal device 200 is doing an aerobic exercise when a change amount D1 of the myopotential integrated value, which will be described later, is larger than 0.

In the example of FIG. 6, it is proved that the user of the portable terminal device 200 can do an aerobic exercise by doing an exercise with an exercise intensity of 2.0 [Mets] or higher, namely, 2.5 [Mets]. A boundary of the exercise intensity, at which an aerobic exercise can be done in this way, is referred to as an “aerobic exercise load limit”. The CPU 201 can estimate the aerobic exercise load limit based on an exercise intensity of an exercise when the exercise is determined to be an aerobic exercise at present or in the past.

Additionally, the CPU 201 calculates an average value of an exercise intensity per unit time, which is calculated by the subprocessor 204, as will be described later. Then, the CPU 201 determines the presence/absence of the fatigue of the body based on largeness/smallness of the myopotential integrated value, and a change amount of the myopotential integrated value if the change amount D2 of the exercise intensity, which will be described later, is within a certain range, equal to or smaller than a threshold value I in this embodiment. As the threshold value I, a positive real number is available.

The reason why the change amount D2 of the exercise intensity is limited to the certain range is to exclude a case where an exercise intensity suddenly changes. Accordingly, the threshold value I can be set based on, for example, an allowable maximum change amount of an exercise intensity. “fatigue of the body” in this embodiment means fatigue of muscles, and can include a state where lactic acid starts to accumulate in muscles due to muscle actions.

Additionally, when the body is getting fatiguing, an amplitude of a potential of muscle actions decreases and an increment of the myopotential integrated value becomes small or reduces even though an exercise is done with the same exercise intensity. Therefore, the CPU 201 according to this embodiment determines that the body is fatigued when a sum S of the myopotential integrated value, which will be described later, is equal to or smaller than a threshold value A and the change amount D1 of the myopotential integrated value is equal to or smaller than a threshold value B.

A predetermined positive real value is available as the threshold value A. Moreover, a predetermined real value is available as the threshold value B. If the change amount D1 of the myopotential integrated value indicates a negative real value, this means that the myopotential integrated value decreases with time. Accordingly, if the change amount D of the myopotential integrated value indicates a negative real value, which is extremely smaller than 0, the user of the portable terminal device 200 has started to feel fatigued in some cases. Accordingly, by setting a positive real value or negative real value close to 0 as the threshold value B, the portable terminal device 200 can detect the fatigue of the body before the user of the portable terminal device 200 starts to feel fatigued.

The myopotential sensor 202 can include electrodes 301 connected via a signal line 302 as illustrated in FIG. 3 to be described later although they are not illustrated in FIG. 2. The myopotential sensor 202 outputs a myopotential of a surface of the body, which makes contact with the electrodes 301, to the subprocessor 204 via the electrodes 301.

The acceleration sensor 203 is a device for measuring an acceleration of the portable terminal device 200 mounted on the body, namely, an acceleration applied to the body. The subprocessor 204 calculates an exercise intensity and an action quantity by using the acceleration measured by the acceleration sensor 203 as will be described later.

The subprocessor 204 notifies the CPU 201 of a myopotential after the subprocessor 204 performs a specified operation such as converting a myopotential output by the myopotential sensor 202 into digital data. Moreover, the subprocessor 204 notifies the CPU 201 of calculation results after the subprocessor 204 calculates an exercise intensity and an action quantity based on the acceleration in a particular direction, for example, the gravity direction, which is measured by the acceleration sensor 203.

In this embodiment, the subprocessor 204 can obtain the exercise intensity by using a relational expression, illustrated in FIG. 7, between an acceleration and an exercise intensity as follows. The relational expression illustrated in FIG. 7 can be represented with the following equations.

(exercise intensity[Mets])=k1×(acceleration a1), 0≦a1<at (1)

(exercise intensity[Mets])=k2×(acceleration a2), at≦a2 (2)

Proportional constants k2, k2, a1, a2, and at are predetermined real values. These constants can be obtained by statistically approximating a relationship between an exercise intensity and an acceleration in a particular direction, for example, the gravity direction, which is applied to the body at the time of the exercise with the exercise intensity when the exercise, such as running, is done with a plurality of exercise intensities.

The subprocessor 204 calculates an action quantity at a time Δt[s] with the following expression (3) after the subprocessor 204 calculates the exercise intensity based on the acceleration measured by the acceleration sensor 203 with the use of the expressions (1) and (2).

(action quantity[Mets·h])=(exercise intensity[Mets])×Δt÷(60×60) (3)

The exercise intensity calculation method according to this embodiment is not limited to the above described one as a matter of course. For example, an exercise intensity corresponding to an acceleration obtained from the acceleration sensor 203 may be stored in the memory 205 as a table. Alternatively, an exercise intensity may be calculated by using information other than an acceleration.

The memory 205 is a volatile storage device for temporarily storing a program and data, which the portable terminal device 200 needs to operate, and part or the whole of a program for implementing the state determination process according to this embodiment. As the memory 205, for example, a RAM (Random Access Memory) or the like is available.

The flash memory 206 is a nonvolatile storage device for storing the program for implementing the state determination process according to this embodiment in addition to the program and the data, which the portable terminal device 200 needs to operate.

The LCD 207 is a display device for displaying data and the like output by the CPU 201 or the like, for example, data illustrated in FIGS. 8 and 9. The touch panel 208 is data input means from an outside. For example, upon detection of a specified operation that a user performs on the touch panel 208, the CPU 201 starts the state determination process according to this embodiment.

Additionally, the portable terminal device 200 can include a wireless communication device 209, a microphone 210, a speaker 211, and Bluetooth (registered trademark) 212.

The wireless communication device 209 is, for example, a communication device for communicating with another portable terminal device by making a wireless communication with a base station that connects to a specified communication network. For example, the wireless communication device 209 outputs, to the speaker 211, voice data generated by executing processes for transmitting, to the other portable terminal device, voice data of a voice input to the microphone 210, and for decoding the signal received from the other portable terminal device.

The microphone 210 is a device for outputting an input voice or the like as a voice signal. Moreover, the speaker 211 is a reproduction device for reproducing a voice or the like from voice data which is received from another portable terminal device and for which processes such as decoding and the like are executed.

As a storage medium readable by the portable terminal device 200, such as the memory 205, the flash memory 206 or the like, a non-transitory medium is available.

Additionally, FIG. 2 depicts the configuration example in the case where the state determination device according to this embodiment is implemented with the portable terminal device 200 such as a cellular phone or the like. This is merely one example. Accordingly, to implement the state determination device according to this embodiment, unneeded components may be suitably removed from among the components illustrated in FIG. 2, or a new component, not illustrated in FIG. 2, may be added.

FIG. 3 is an external view of the portable terminal device 200. In the portable terminal device 200, the signal line 302 connecting to the myopotential sensor 200 within the portable terminal device 200 is connected to the electrodes 301 as illustrated in FIG. 3. The plurality of electrodes 301 are attached to an inside of a band 303 made of an elastic material. The band 303 is attached to an arm or the like of a user to make contact with a skin surface of the user, so that the myopotential sensor 202 can measure a myopotential via the electrodes 301 in contact with the skin surface of the user.

The electrodes 301, the signal line 302, and the band 303, which are illustrated in FIG. 3, are merely examples used in this embodiment. Accordingly, the arrangement and the shape of the electrodes 301, and the connection with the myopotential sensor 202 are not limited to those illustrated in FIG. 3. For example, the myopotential sensor 202 may be attached to the band 303 as illustrated in FIG. 10. In this case, by attaching a Bluetooth transmitter to the band 303 along with the myopotential sensor 202, the Bluetooth transmitter can transmit the myopotential that the myopotential sensor 202 measures via the electrodes 301 to the portable terminal device 200. Moreover, the electrodes 301 may be provided on a right side surface and a left side surface of the portable terminal device 200 as illustrated in FIG. 11.

FIGS. 4 and 5 are flowcharts illustrating the state determination process of the body, which is executed by the portable terminal device 200 according to this embodiment.

Upon detection of a specified operation of a user on the touch panel 208, the portable terminal device 200 starts the state determination process (step S401).

The portable terminal device 200 measures a myopotential and an exercise intensity for one minute at intervals of one second (steps S401 and S402, “NO” in step S403). The portable terminal device 200 can obtain the myopotential from the myopotential sensor 202. Moreover, the portable terminal device 200 can obtain, from the subprocessor 204, an exercise intensity calculated based on an acceleration obtained from the acceleration sensor 203 as described above with reference to FIG. 2.

After the portable terminal device 200 measures the myopotential and the exercise intensity for one minute (“YES” in S403), the portable terminal device 200 shifts the process to step S404. If a stop request of the state determination process is issued with a specified operation of the user (“YES” in step S404), the portable terminal device 200 terminates the state determination process (step S419).

If the stop request of the state determination process is not issued (“NO” in step S404), the portable terminal device 200 calculates a myopotential integrated value (μV·s) by integrating the myopotential measured for one minute in step S401 with time (step S405). Then, the portable terminal device 200 calculates a sum S of the myopotential integrated value calculated at this time (hereinafter referred to as a first myopotential integrated value) in step S405 and a myopotential integrated value calculated at the preceding time (hereinafter referred to as a second myopotential integrated value) in step S405 (step S406).

Adopting the sum S in this embodiment is to improve accuracy of the determination of the presence/absence of fatigue, which uses the myopotential integrated value, by reducing an extreme influence on a value of the myopotential with the use of the myopotential integrated value obtained for the longest possible period. Accordingly, not only the sum of the myopotential integrated value calculated at this time and that calculated at the preceding time but a sum of the myopotential integrated value calculated at this time, that calculated at the preceding time, and that calculated at time antecedent to the preceding time in step S405 is available.

Additionally, the portable terminal device 200 calculates the change amount D1 of the first myopotential integrated value from the second myopotential integrated value (step S407).

Furthermore, the portable terminal device 200 calculates an average value of the exercise intensity measured for one minute in step S402 (step S408). Then, the portable terminal device 200 calculates the change amount D2 of the average value of the exercise intensity, which is hereinafter referred to as a first exercise intensity and calculated at this time in step S408, from an average value of the exercise intensity, which is hereinafter referred to as a second exercise intensity and calculated at the preceding time in step S408 (step S409).

If the change amount D1 of the myopotential integrated value calculated in step S407 is larger than 0 in step S410 (“YES” in step S410), the portable terminal device 200 determines that the current exercise is an aerobic exercise (step S411). Then, the portable terminal device 200 stores the average value of the exercise intensity calculated in step S408, namely, the first exercise intensity at a specified address of the flash memory 206.

Additionally, if the first exercise intensity obtained when the exercise is determined as the aerobic exercise in step S411 is a minimum value among average values of the exercise intensity when the exercise is determined as the aerobic exercise (“YES” in step S412), the portable terminal device 200 shifts the process to step S413. In this case, the portable terminal device 200 estimates the average value of the exercise intensity of the exercise when the exercise is determined as the aerobic exercise in step S411, namely, the first exercise intensity as an aerobic exercise load limit (step S413). Accuracy of this estimation can be improved by repeating the process of step S413, namely, by user doing an exercise with diverse exercise intensities with the use of the portable terminal device 200.

If the change amount D2 of the exercise intensity, which is calculated in step S409, is equal to or lower than the threshold value I in step S414 (“YES” in step S414), the portable terminal device 200 shifts the process to step S415. In this case, the sum S of the myopotential integrated values, which is calculated in step S406, is equal to or smaller than the threshold value A (“YES” in step S415) and the change amount D1 of the myopotential integrated value, which is calculated in step S407, is equal to or smaller than the threshold value B (“YES” in step S416), the portable terminal device 200 determines that the body is fatigued (step S417). Then, the portable terminal device 20 displays determination results of steps S410 to S417 on a display screen of the portable terminal device 200 (step S418).

For example, the portable terminal device 200 can notify a user of the process results in steps S410 to S413 by displaying a display screen illustrated in FIG. 8. In the example of FIG. 8, a user is notified of an aerobic exercise currently being done, the current action quantity, and the aerobic exercise load limit. As the current action quantity, an action quantity (Mets·h) obtained by using the expression (3) based on the exercise intensity calculated in step S402 is available. Moreover, as the aerobic exercise load limit, an action quantity (Mets·h) obtained by using the expression (3) based on the first exercise intensity estimated as the aerobic exercise load limit in step S413 is available.

Additionally, for example, the portable terminal device 200 can notify a user of the process results in steps S414 to S417 by displaying a display screen illustrated in FIG. 9. In the example of FIG. 9, it is notified to a user that his or her body is fatigued.

The displays illustrated in FIGS. 8 and 9 may be displayed on one display screen as a matter of course. Moreover, in the flowchart of FIG. 5, the portable terminal device 200 displays the determination results of steps S410 to S417 in step S418 only if the body is determined to be fatigued. However, the portable terminal device 200 may display the process results of steps S410 to S413, for example, each time the process of step S413 is executed.

As described above, the portable terminal device 200 measures a myopotential from a skin surface of the body. Then, the portable terminal device 200 calculates a myopotential integrated value based on the myopotential measured per unit time, for every one minute in this embodiment. Then, the portable terminal device 200 determines that the body is fatigued if the sum S of the myopotential integrated values is equal to or smaller than the threshold value A and the change amount D1 of the myopotential integrated value is equal to or smaller than the threshold value B (“YES” in step S415, “YES” in step S416).

In this way, the portable terminal device 200 can determine fatigue of the body based on a myopotential of a skin surface of the body. Accordingly, components of blood do not need to be analyzed in order to determine the fatigue of the body. Accordingly, with the portable terminal device 200, a state of the body can be easily determined.

Additionally, the portable terminal device 200 determines that the body is doing an aerobic exercise if the change amount of the myopotential integrated value calculated per unit time, for every one minute in this embodiment is larger than 0 (“YES” in step S410). Accordingly, with the portable terminal device 200, whether or not the body is doing an aerobic exercise can be determined without performing special operations such as an analysis of components of blood.

Furthermore, the portable terminal device 200 determines the fatigue of the body based on a myopotential integrated value calculated based on a myopotential measured per unit time and the change amount of the myopotential integrated value, whereby the portable terminal device 200 can determine the fatigue of the body in a short time, for example, two minutes at the shortest in the case where the unit time is one minute. Similarly, the portable terminal device 200 can determine, in a short time, whether or not the body is doing an aerobic exercise.

The above described state determination process can be implemented by using, for example, an information processing device (computer) illustrated in FIG. 12. The information processing device illustrated in FIG. 12 includes a CPU (Central Processing Unit) 1201, a memory 1202, an input device 1203, an output device 1204, an external recording device 1205, a medium driving device 1206, and a network connection device 1207. These components are interconnected by a bus 1208.

The memory 1202 is a semiconductor memory such as a ROM (Read Only Memory), a RAM (Random Access Memory), a flash memory, or the like, and stores a program and data, which are used for the state determination process. For example, the CPU 1201 executes the above described state determination process by executing the program with the use of the memory 1202.

The input device 1203 is, for example, a keyboard, a pointing device, or the like, and is used to input an instruction and information from an operator. The output device 1204 is, for example, a display device, a printer, a speaker, or the like, and used to output an inquiry or a process result to an operator.

The external recording device 1205 is, for example, a magnetic disk device, an optical disk device, a magneto-optical disk device, a tape device, or the like. The external recording device 1205 also includes a hard disk drive. The information processing device stores a program and data in the external recording device 1205, and can use the program and the data by loading them into the memory 1202.

The medium driving device 1206 drives a portable recording medium 1209, and accesses recorded contents of the medium. The portable recording medium 1209 is a memory device, a flexible disk, an optical disk, a magneto-optical disk, or the like. The portable recording medium 1209 also includes a Compact Disk Read Only Memory (CD-ROM), a Digital Versatile Disk (DVD), a Universal Serial Bus (USB) memory, and the like. An operator stores a program and data on the portable recording medium 1209, and can use the program and the data by loading them into the memory 1202.

As described above, a computer-readable recording medium storing a program and data, which are used for the state determination process, includes a physical (non-transitory) recording medium such as the memory 1202, the external recording device 1205, or the portable recording medium 1209.

The network connection device 1207 is a communication interface, connected to a communication network 1210, for performing a data conversion accompanying a communication. The information processing device receives the program and the data from an external device via the network connection device 1207, and can use the program and the data by loading them into the memory 1202.

The disclosed embodiments and advantages thereof have been described in detail. A person having ordinary skill in the art could make various changes, additions, and omissions in a scope that does not depart from the gist of the present invention explicitly recited in claims.

According to embodiments of this state determination device, a state determination device that can easily determine a state of the body can be provided.

All examples and conditional language recited herein are intended for pedagogical purposes to aid the reader in understanding the invention and the concepts contributed by the inventor to furthering the art, and are to be construed as being without limitation to such specifically recited examples and conditions, nor does the organization of such examples in the specification relate to a showing of the superiority and inferiority of the invention. Although the embodiments of the present invention have been described in detail, it should be understood that the various changes, substitutions, and alterations could be made hereto without departing from the spirit and scope of the invention.

What is claimed is:

1. A state determination device, comprising:
a myopotential measurement unit configured to measure a myopotential of a surface of a body;
a myopotential integrated value calculation unit configured to calculate a myopotential integrated value by integrating, with time, the myopotential measured by the myopotential measurement unit for a measurement period at predetermined measurement intervals; and
a fatigue determination unit configured to determine presence/absence of fatigue of the body based on a size of the myopotential integrated value calculated by the myopotential integrated value calculation unit, and a change amount of the myopotential integrated value.

2. The state determination device according to claim 1, further comprising
an aerobic exercise determination unit configured to determine whether or not the body is doing an aerobic exercise based on presence/absence of a change of the myopotential integrated value calculated by the myopotential integrated value calculation unit.

3. The state determination device according to claim 1, wherein
the fatigue determination unit determines the presence/absence of the fatigue of the body based on a change amount of a first myopotential integrated value calculated by the myopotential integrated value calculation unit based on the myopotential measured by the myopotential measurement unit for a first measurement period from a second myopotential integrated value calculated by the myopotential integrated value calculation unit based on a myopotential measured by the myopotential measurement unit for a second measurement period, which is a measurement period immediately preceding the first measurement period, and based on a sum of the first myopotential integrated value and the second myopotential integrated value.

4. The state determination device according to claim 3, wherein
the fatigue determination unit determines that the body is fatigued when the change amount of the first myopotential integrated value from the second myopotential integrated value is equal to or smaller than a particular threshold value, and the sum of the first myopotential integrated value and the second myopotential integrated value is equal to or smaller than a particular threshold value.

5. The state determination device according to claim 2, wherein
the aerobic exercise determination unit determines whether or not the body is doing an aerobic exercise based on presence/absence of a change amount of a first myopotential integrated value calculated by the myopotential integrated value calculation unit based on the myopotential measured by the myopotential measurement unit for a first measurement period from a second myopotential integrated value calculated by the myopotential integrated value calculation unit based on the myopotential calculated by the myopotential calculation unit for a second measurement period, which is a measurement period immediately preceding the first measurement period.

6. The state determination device according to claim 5, wherein
the aerobic exercise determination unit determines that the body is doing an aerobic exercise when the change amount of the first myopotential integrated value from the second myopotential integrated value is larger than 0.

7. The state determination device according to claim 2, further comprising:
an exercise intensity measurement unit configured to measure an exercise intensity of the body; and
an exercise intensity calculation unit configured to calculate an exercise intensity for the measurement period by calculating an average value of the exercise intensity measured by the exercise intensity measurement unit for the measurement period at the measurement intervals, wherein
the aerobic exercise determination unit stores, in a storage unit, the exercise intensity calculated by the exercise intensity calculation unit when the aerobic exercise determination unit determines that the body is doing an aerobic exercise, and estimates a minimum exercise intensity among exercise intensities stored in the storage unit as an aerobic exercise load limit, which is a lower limit of the exercise intensity with which the aerobic exercise can be done.

8. A state determination method for determining a state of a body, comprising:
measuring a myopotential of a surface of the body;
calculating a myopotential integrated value by integrating, with time, the myopotential measured for a measurement period at predetermined measurement intervals; and
determining presence/absence of fatigue of the body based on a size of the calculated myopotential integrated value, and the change amount of the myopotential integrated value.

9. The state determination method according to claim 8, further comprising
determining whether or not the body is doing an aerobic exercise based on presence/absence of a change of the calculated myopotential integrated value.

10. A non-transitory computer-readable recording medium having stored therein a program for causing an information processing device to execute a determination process for determining a state of a body, the determination process comprising:
measuring a myopotential of a surface of the body;
calculating a myopotential integrated value by integrating, with time, the myopotential measured for a measurement period at predetermined measurement intervals; and
determining presence/absence of fatigue of the body based on a size of the calculated myopotential integrated value, and a change amount of the myopotential integrated value.

11. The medium according to claim 10, the determination process further comprising
determining whether or not the body is doing an aerobic exercise based on presence/absence of a change of the calculated myopotential integrated value.
